(19) United States
(12) Patent Application Publication
(10) Pub. No.: US 2025/0295082 A1
(43) Pub. Date: Sep. 25, 2025

(54) LETTUCE PLANT RESISTANT TO DOWNY MILDEW AND RESISTANCE GENE

(71) Applicant: Enza Zaden Beheer B.V., Enkhuizen (NL)

(72) Inventors: Bas Ter Riet, Medemblik (NL); Mathieu André Pel, Enkhuizen (NL)

(21) Appl. No.: 18/869,024

(22) PCT Filed: Jun. 3, 2022
(86) PCT No.: PCT/EP2022/065237

§ 371 Date: Nov. 25, 2024

(51) Int. Cl. A01H 1/00; A01H 5/12; A01H 6/14; C07K 14/415

(52) U.S. Cl. CPC A01H 1/1255; A01H 5/12; A01H 6/1472; C07K 14/415

(57) ABSTRACT

The present invention relates to a lettuce plant that is resistant to downy mildew, more specifically to a lettuce plant that comprises a resistance gene that confers broad spectrum resistance to oomycetes in lettuce, more specifically B. lactucae. Furthermore, the present invention relates to a resistance gene and a method for obtaining a lettuce plant that is resistant to downy mildew, wherein the method comprises the step of introducing said resistance gene into a lettuce plant.

The present invention relates to a lettuce plant that is resistant to downy mildew, more specifically to a lettuce plant that comprises a resistance gene that confers broad spectrum resistance to oomycetes in lettuce, more specifically Bremia lactucae. Furthermore, the present invention relates to a resistance gene and a method for obtaining a lettuce plant that is resistant to downy mildew, wherein the method comprises the step of introducing said resistance gene into a lettuce plant.

Downy mildew refers to several types of oomycete microbes that are pathogens of plants. Downy mildew can originate from various species, but mainly of Peronospora, Plasmopara and Bremia. Downy mildew is a problem in many food crops, for example in lettuce caused by B. lactucae, affecting the production of this crop worldwide. Plants that are being affected include food crops such as brassicas (e.g. cabbage), grape, spinach, lettuce, onion, and cucumber. Downy mildew infection shows symptoms of discoloured areas on upper leaf surfaces in combination with white, grey or purple mould located on the lower side of the leaf facing the floor. Disease is spread from plant to plant by airborne spores.

Lettuce, mostly known as Lactuca sativa, but also including Lactuca species such as L. serriola, L. saligna or L. virosa, is a very important crop worldwide. Some of the most popular varieties available belong to the Iceberg, Romaine, Butterhead, Batavia and Oakleaf lettuce types. There are many plant pathogens that affect L. sativa, and some of the diseases caused by these pathogens are downy mildew, sclerotinia rot, powdery mildew, fusarium wilt of which the most important disease is lettuce downy mildew, which is caused by the B. lactucae, an oomycete pathogen that belong to Peronosporaceae.

For some vegetable crops, such as lettuce, cultivars with resistance to downy mildew are available. However, the pathogen under pressure will mutate to break down the disease resistance and new disease resistance in crops is needed to control infection. Especially in lettuce the occurrence of downy mildew resistance is particularly complex as there are many different races, and new downy mildew resistant species emerging all the time, as found in European and the USA markets.

In lettuce, infection of B. lactucae result in yellow to pale green lesions that eventually become necrotic due to secondary pathogens leading to major crop losses. Fungicides can be used to control B. lactucae, but eventually B. lactucae becomes immune to these chemicals, because over time the pathogen also acquires resistance to fungicides. Furthermore, there are multiple lettuce varieties available that are resistant to B. lactucae but resistance is quickly overcome because new Bremia races develop rapidly. Therefore, it is of the utmost importance to find other methods to control B. lactucae infection. Most preferably is to identify a resistance gene that gives broad resistance against B. lactucae and to provide for lettuce plants that are resistant to downy mildew. Therefore, identification of resistance genes is a promising alternative.

During the development of new disease or pathogen resistant plants often traits are being combined, for example by introgression of a genetic locus comprising one or more resistance gene, thereby combining multiple resistance genes to combat the pathogen being able to overcome the resistance. However the introgression fragments often comprise, apart from the resistance gene of interest, other genetic elements that may negatively affect the plant in terms of yield, growth, vitality, and seed production. For example introgression of (additional) new resistance genes in lettuce often result in a severe reduction in seed production as a result of linkage drag. Therefore, apart from the generation of improved disease resistance in plant, there is also great benefit to reduce this so called linkage drag which becomes an increasing hurdle in plant breeding. Single event introgression as well as marker-aided selection techniques in the flanking regions of the resistance gene to reduce the introgression segment can play an important role here, with the objective of minimizing residual genetics (apart from the resistance gene) being transferred to the parent plant and to eliminate linkage drag effects. Gene editing and the use of sequence information in genome-wide selection will further add to the precision of reduction of linkage drag.

Considering the above, there is a need in the art to provide plants that are resistant to downy mildew and wherein plants have a broad-spectrum resistance against this pathogen. Furthermore, it is an object of present invention to provide plants having a broad-spectrum downy mildew resistance, and to provide a method to obtain such downy mildew resistant plants.

It is an object of the present invention, amongst other objects, to address the above need in the art. The object of present invention, amongst other objects, is met by the present invention as outlined in the appended claims.

Specifically, the above object, amongst other objects, is met, according to a first aspect, by the present invention by a downy mildew resistant lettuce plant, wherein said lettuce plant comprises a V10 resistance gene encoding a protein having at least 90%, preferably at least 95%, more preferably at least 98% even more preferably 99%, most preferably 100% sequence identity with amino acid sequence of SEQ ID No. 2 providing downy mildew resistance, wherein said lettuce plant is resistant to B. lactucae races Bl:16 to Bl:37. The downy mildew resistance conferring gene V10 is a dominant resistance trait, and may be homozygous or heterozygous present in a downy mildew resistant lettuce plant. The resistance gene against B. lactucae has been found on chromosome 1 in lettuce, present in the Major Resistance Cluster 1. This V10 resistance gene of the present invention gives resistance to B. lactucae races Bl:16-37EU and preferably also Bl:1-9US wherein said Bremia races have been characterized and classified according to the SEXTET code by IBEB (International Bremia Evaluation Board). Furthermore, previous disease resistance tests show that the V10 resistance gene further provides resistance to the “older”, less recent Bremia races Bl:1 to Bl:15. Therefore the V10 resistance gene provides full spectrum resistance to Bl:1-37EU.

The majority of disease resistance genes in plants encode nucleotide-binding site leucine-rich repeat proteins, also known as NBS-LRR proteins (encoded by R genes). These proteins are characterized by nucleotide-binding site (NBS) and leucine-rich repeat (LRR) domains as well as variable amino- and carboxy-terminal domains and are involved in the detection of diverse pathogens, including bacteria, viruses, fungi, nematodes, insects and oomycetes. There are three major subfamilies of plant NBS-LRR proteins defined by the Toll/interleukin-1 receptor (TIR) also called TNLs, the coiled-coil (CC) motifs in the amino-terminal domain containing NBS-LRRs also called CNLs and RPW8-NLTRs also called RNLs. A typical R gene contains an NB-ARC domain which is proposed to regulate activity of the R protein.

The V10 resistance gene is unique since it comprises features that are typical for plant resistance gene, more specifically the V10 resistance gene comprises a TIR domain, however it does not comprise a nucleotide binding site which is generally present in resistance genes, and it has LRR domains. The V10 gene is initially picked up by fine mapping and VIGS experiments based on the L. sativa genome. The majority of disease resistance genes in plants encode nucleotide-binding site leucine-rich repeat proteins, also known as NBS-LRR proteins (encoded by R genes). These proteins are characterized by nucleotide-binding site (NBS) and leucine-rich repeat (LRR) domains as well as variable amino- and carboxy-terminal domains and are involved in the detection of diverse pathogens, including bacteria, viruses, fungi, nematodes, insects, and oomycetes. The V10 gene comprises a TIR domain and several LRR regions (but no NBS) within the gene that may be beneficial and contributes to generate a new R gene that confers broad spectrum Bremia resistance.

The presence of the V10 resistance gene will provide broad spectrum Bremia resistance to lettuce plants. To decrease the chances of the pathogen overcoming the resistance, as often seen with R genes, multiple R genes can be combined to enhance the durability of disease resistance. For example, the downy mildew resistant lettuce plant of the present invention may further comprise one or more resistance genes located on chromosome 1 at a significant distance from the V10 resistance gene or with R genes located at different linkage groups. Additionally or alternatively the V10 resistance gene may be stacked with other resistance genes on other chromosomes. V10 is present on the MRCI cluster (major resistance cluster 1) and could be used to combine with another active Bremia resistance gene. As such, stacking of multiple resistance genes will enable broad and durable Bremia resistance in lettuce.

To demonstrate that the V10 resistance gene provides Bremia resistance, this V10 resistance gene was silenced by tobacco rattle virus (TRV)-based virus-induced gene silencing (VIGS) to induce susceptibility to B. lactucae infection in resistant L. sativa lines containing the V10 resistance gene. With VIGS it was demonstrated that the V10 resistance gene was associated with downy mildew resistance, since VIGS induced gene silencing was used to create Bremia susceptibility in resistant Lactuca accessions containing only V10 resistance. Resistant lettuce plants were transiently transformed with a silencing construct specific against the V10 resistance gene which resulted in the silencing of the resistance gene and as a consequence made the plant or plant organs susceptible to B. lactucae infection, thus by “removing” or silencing the V10 resistance gene via virus induced gene silencing.

According to another preferred embodiment, the present invention relates to the lettuce plant, wherein said V10 resistance gene comprises a coding sequence having at least 90%, preferably at least 95%, more preferably at least 98%, most preferably 100% sequence identity with SEQ ID No. 1. Said V10 resistance gene is preferably located on chromosome 1 of the lettuce plant.

According to a preferred embodiment, the present invention relates to the lettuce plant, wherein the lettuce plant is further resistant to one or more of B. lactucae races selected from the group consisting of races Bl:1-15EU. Recent experiments show that a lettuce plant of the present invention comprising the V10 resistant gene is resistant to Bremia races from Bl:16 to Bl:37EU. Previous disease resistance tests on less recent Bremia races Bl:115EU (data not shown) show that the V10 resistance gene further provides resistance to these Bremia races. Resistance to B. lactucae in the lettuce of present invention comprises full spectrum resistance to B. lactucae races Bl:1 to Bl:37EU.

According to yet another preferred embodiment, the present invention relates to the lettuce plant, wherein the plant is selected from Lactuca sativa, Lactuca virosa, Lactuca saligna, Lactuca serriola, Lactuca aculeate, Lactuca georgica, Lactuca perennis, Lactuca tatarica, Lactuca viminea, preferably Lactuca sativa.

According to a preferred embodiment, the present invention relates to the lettuce plant, wherein the V10 resistance gene is at least heterozygous present in the lettuce plant, preferably homozygous present.

According to yet another preferred embodiment, the present invention relates to the lettuce plant, wherein the V10 resistance gene is obtainable, derived, or originates from a lettuce plant of L. virosa. Most preferably the present invention relates to the lettuce plant, wherein the V10 resistance gene is obtainable, derived, or originates from a lettuce plant deposited under number NCIMB 42786.

The present invention, according to a second aspect, relates to seed of a lettuce plant of the present invention, comprising a V10 resistance gene encoding a protein as described above. The seed comprises the V10 resistance gene as described above.

According to a preferred embodiment, the present invention relates to a resistance gene that confers resistance to B. lactucae in lettuce plants, wherein the resistance gene encodes for a protein that has at least 90%, preferably at least 95%, more preferably at least 98%, most preferably 100% sequence identity with SEQ ID No. 2. The V10 resistant gene is a dominant trait.

According to another preferred embodiment, the present invention relates to a resistance gene that confers resistance to B. lactucae in lettuce plants, wherein the coding sequence of said resistance gene has at least 90%, preferably at least 95%, more preferably at least 98%, most preferably 100% sequence identity with SEQ ID No. 1.

According to yet another preferred embodiment, the present invention relates to a resistance gene that confers resistance to B. lactucae in lettuce plants, wherein the resistance gene provides resistance to at least Bremia lactucae races Bl:16-37EU, preferably Bremia lactucae races Bl:1-37EU in lettuce. The resistance gene preferably further provides resistance to B. lactucae US spectrum Bl:1-9US.

According to yet another preferred embodiment, the present invention relates to the resistance gene that confers resistance to B. lactucae in lettuce plants, wherein the plant is selected from Lactuca sativa, Lactuca virosa, Lactuca saligna, Lactuca serriola, Lactuca aculeate, Lactuca georgica, Lactuca perennis, Lactuca tatarica, Lactuca viminea, preferably Lactuca sativa.

The present invention, according to a further aspect, relates to a method for identifying (i) a downy mildew resistant lettuce plant of the present invention or (ii) a seed from said plant wherein the method comprises the step of establishing, in the genome of a plant or seed the presence of a V10 resistance gene encoding a protein as defined above. The step of establishing, in the genome of the seed, the presence of any genetic information, including the presence of the V10 resistance gene encoding the protein as defined above, may suitably involve allowing the seed to grow into a plant and establishing the presence of the genetic information in the genome of the plant grown from the seed.

According to yet another preferred embodiment, the present invention relates to the method for identifying a downy mildew resistant lettuce plant of present invention, wherein the step of establishing, comprises establishing the presence of SEQ ID No. 1, and/or SEQ ID No. 3 in combination with SEQ ID No. 4, most preferably SEQ ID No.1 in combination with SEQ IDs No. 3 and 4. A downy mildew resistant plant of present invention comprising the V10 resistance gene can be identified by the presence in the genome of said plant of one or more sequences selected from the group consisting of, SEQ ID No. 1, SEQ ID No. 3, SEQ ID No. 4, preferably only SEQ ID No. 1, or SEQ ID No. 1 and SEQ ID No. 3, or SEQ ID No. 1 and SEQ ID No.4, or SEQ ID No. 1 and SEQ ID No. 3 and 4. Presence of the V10 is identified by SEQ ID No. 1. Most preferably the plant can be identified by the presence of SEQ ID No.1 in combination with SEQ IDs No. 3 and 4.

The present invention, according to a further aspect, relates to a method for obtaining a lettuce plant that is resistant to downy mildew, wherein the method comprises the steps of,
a) crossing a lettuce plant comprised of the resistance gene of the present invention with a lettuce plant susceptible to downy mildew and which does not comprise said resistance gene,
b) optionally, selfing the plant obtained in step a) for at least one time,
c) selecting the plants that are resistant to downy mildew.

A plant having this resistant phenotype can be obtained via use of gene editing and/or mutation techniques, such as EMS mutagenesis or CRISPR/Cas in concert with cloning techniques on the V10 resistance gene to generate disease resistant crops. A resistance gene can be brought into the plant by known means including e.g. transgenic techniques or by introgression, wherein the resistance providing sequence(s) are introduced into the plant.

The present invention, according to a further aspect, relates to the use of a gene construct or plasmid for introducing a resistance gene into the genome of a plant or plant cell and providing broad spectrum resistance to downy mildew caused by one or more of B. lactucae races selected from the group of race Bl:16-37EU wherein the gene construct is comprised of the resistance gene operably linked to expression providing sequences in said plant. The resistance gene of present invention may be transferred (e.g. by transformation or transfection) into plants, such as lettuce plants, using a plasmid or vector or linear gene construct that comprises the resistance gene of present invention. The V10 resistance gene, after being transferred into the lettuce plant will provide resistance to B. lactucae, i.e. resistance to at least B. lactucae of race Bl:16-37EU, preferably Bl:1-37EU.

The present invention will be further detailed in the following examples and figures wherein:

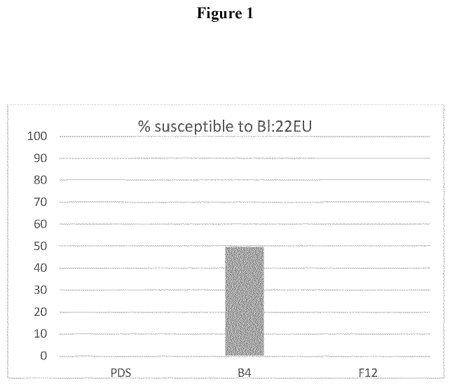

FIG. 1: shows the % of susceptible leaves of lettuce that have been infected with Bremia lactucae Bl:22EU, after VIGS silencing of the V10 resistance gene of present invention of a lettuce plant of present invention comprising the V10 resistance gene using VIGS gene silencing constructs of Table 2 and subsequently infected with B. lactucae. As expected with transient gene silencing, VIGS gene silencing does not result in fully 100% silencing of the gene in all plants. However, the leaves from plants wherein the V10 resistance gene has been silenced by VIGS silencing (B4 silencing construct), showed a significant percentage of susceptible leaves (about 50%) when infected with Bremia as compared to plants where the V10 gene was not silenced (i.e. by F12 or PDS silencing construct on the plant comprising the V10 gene).

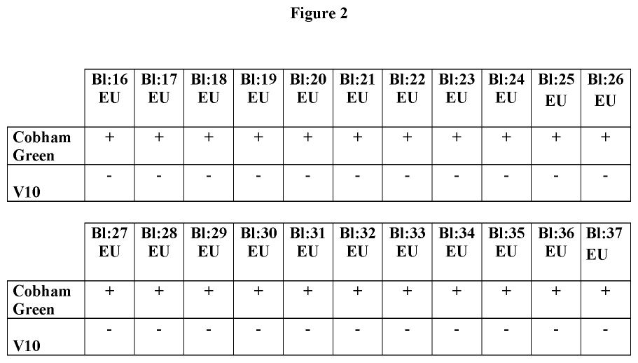

FIG. 2: shows an overview of the disease test performed with the most recent isolates of B. lactucae Bl:16-37EU on L. sativa lines Cobham Green, and the plant of present invention comprising the V10 resistance gene. The plant of present invention shows to be resistant to all tested downy mildew isolates, Bl:16-37EU, providing broad spectrum resistance.

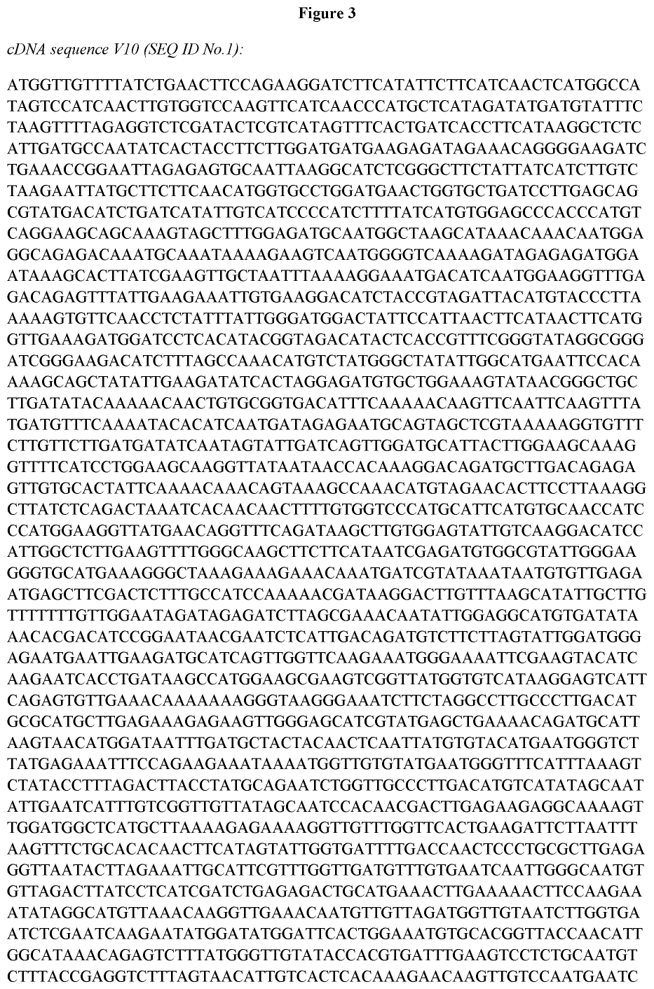

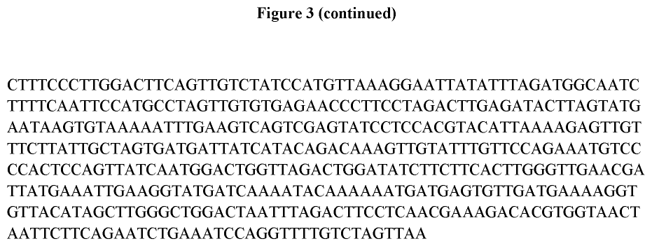

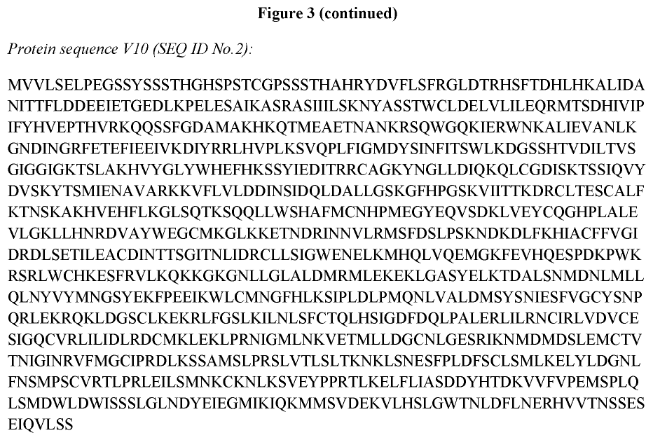

FIG. 3: shows the coding (cDNA) sequence of the V10 resistance gene (SEQ ID No. 1) and its protein sequence (SEQ ID No.2).

### EXAMPLES

### Gene Mapping of V10 Resistance Gene in L. virosa

Gene mapping experiments were done to identify a resistance gene that is involved in full spectrum Bremia (B. lactucae) resistance in lettuce (L. sativa). The resistance gene was originally isolated from L. virosa and was mapped on chromosome 1, providing full spectrum Bremia resistance in lettuce.

The identified resistance locus comprises two markers; the marker 1 (SEQ ID No.3) and marker 2 (SEQ ID No.4), providing a resistance locus which comprises a novel resistance gene identified as V10. After fine mapping in a population of about 12,000 plants there was one putative resistance gene present in the identified resistance locus. The SNPs are indicated in bold and underlined on which the resistant plants could be selected. In Bremia susceptible lettuce plants the indicated SNP nucleotide was an “T” in respect to SEQ ID No. 3, and a “G” for SEQ ID No. 4.

TABLE 1
Marker sequences

| Marker | Sequence |
|---|---|
| Marker 1 | ACTAGCATATAAAAAGCATAACAGATAGTGGCATACCAGACAA |
| (SEQ ID No. 3) | TTATCACAAAGACAATCATCTACATATAAACAAGTACTAGTGGG |
| | CCGGCATTGGTGCCTTAGACCCACATCTACCGGA |
| Marker 2 | CCATAAATTCCCCGTCGCCGGGACCTTCCCCATTGATTCAACCCC |
| (SEQ ID No. 4) | AACACCCTATCCCTCATCCACCAATTTCACCACCACCACCACCG |
| | CCACCCATCTCTATACGATCACCGCCTCCTCC |

### V10 Resistance Gene Silencing Experiment Using Virus Induced Gene Silencing (VIGS)

To demonstrate that the V10 resistance gene provides Bremia resistance, the V10 resistance gene was silenced by tobacco rattle virus (TRV)-based virus-induced gene silencing (VIGS) to induce susceptibility to B. lactucae infection in L. sativa lines containing the V10 resistance gene. Tobacco rattle virus (TRV)-derived VIGS vectors have been abundantly described to study gene function in Arabidopsis thaliana, Nicotiana benthamiana, Solanum esculentum and other plants (see for example Huang C, Qian Y, Li Z, Zhou X.: Virus-induced gene silencing and its application in plant functional genomics. Sci China Life Sci. 2012;55(2):99-108). With VIGS it was demonstrated that the V10 resistance gene was associated with downy mildew resistance, since VIGS induced gene silencing was used to create Bremia susceptibility in resistant Lactuca accessions comprising the V10 gene. Resistant lettuce plants were transiently transformed with a silencing construct specific against the resistance V10 gene which will result in the silencing of the resistance gene.

Briefly, lettuce plants containing the V10 resistance gene were silenced for V10 resistance gene by VIGS using different silencing construct to identify if this V10 resistance gene was indeed responsible for the observed resistance. Two VIGS-constructs were used, one (B4) that results in specific silencing of the V10 resistance gene and a control construct (F12) that targets a region on chromosome 1 in close proximity of the V10 resistance gene. Furthermore, independent of resistance gene silencing the PDS gene was silenced as well that served as positive control to indicate if VIGS is working and to determine the efficiency. The PDS gene is involved in carotenoid biosynthesis and is the first step in lycopene biosynthesis. This step is catalyzed by the enzyme phytoene desaturase (PDS). When silencing of the PDS gene is achieved, this results in bleached leaves. Experiments showed bleached leaves indicating that the VIGS silencing was achieved and performed correctly (data not shown). All plants that were VIGS inoculated were harvested and put in a tray and sprayed with Bremia to test the effect of the gene silencing on disease resistance.

The VIGS constructs were cloned in the K20 vector (See Table 2 for sequences, respectively SEQ ID No. 5, SEQ ID No. 6). The constructs were transformed and transiently expressed into a lettuce plant of present invention that is resistant to Bremia, using co-cultivation with agrobacterium (GV3101) to study the resistance gene function in relation to Bremia resistance. The % of susceptible Bremia leaves was observed in both groups and both silencing constructs. With the leaves of VIGS-experiments independent disease tests (see below) were performed to observe that when V10 resistance gene was silenced, plants became susceptible to Bremia.

Results (FIG. 1) indicate that when V10 was silenced by VIGS with the B4 construct the plants became susceptible (49,6% of the leaves showed infection) after Bremia infection (Bl:22EU) confirming that the resistance gene is linked to a resistance gene that provides the plant resistance against Bremia. The PDS and F12 controls plants remained resistant to the Bremia infection, all leaves were unaffected. Identical results were obtained on Bl:31EU and Bl:33EU (results not shown), where silencing of the V10 gene resulted in susceptibility of the lettuce plant.

TABLE 2
VIGS constructs

| VIGS-constructs | Sequence |
|---|---|
| B4 | ACAAAAGCAGCTATATTGAAGATATCACTAGGAGATGTGATGG |
| (SEQ ID No. 5) | AAAGTATATTGGGCTGCTTGATATACAAAAACAACTGTGTGGTG |
| | ATATTTCAAAAACAAGTTCAATTCAAATTTATGATGTTTCAAAA |
| | TACACATCAATGATAGAGAATGCAGTAGCTCGTAAAAGGGTGTT |
| | TATTGTTCTTGATGATATCAATAGTATTGATCAGTTGGATGCATT |
| | ACTTGGAAGCAAAGGTTTTCATCCTGGAAGCAAGGTTATAATAA |
| | CCACAAAGGACAGATGCTTGACAGAGAGTTGTGCAC |
| F12 | TGGCGGCACTACATCCACCATATGTGTTTGTATGCCTCTTCTTCG |
| (SEQ ID No. 6) | TATTTCCAATTCCCACAATCATCTTCCTGATCCCCTTCCTGTTCTT |
| | GGTCGCGCCATTGCTGGTTGCTCCAATCATAACAGCGTCGGAGA |
| | GACCTTAGCCCGAGATACACTGGAACAGGTGATGATGGAAGCC |
| | CTTTCAACGTCAGGCTCAAAGAAATCCGCTGTTCAAGCTGTTTG |
| | TTTAGGCGTATCAGGTGTCAATCATCCA |

### Disease Test and Biotest for Downy Mildew in Lettuce

Leaves of resistant plants transiently transformed with the above described VIGS constructs, were put in trays with moistened paperboard and infected with Bremia race 22. Infected seedlings are suspended in 20 ml water, filtered by cheesecloth and the flow-through is collected in a spray flask. The trays are spray-inoculated with the B. lactucae suspension. The trays are covered with a glass plate and stored in a climate chamber at 15°° C. (12 hours of light). A black, opaque foil is placed over the trays for one day to improve growth of B. lactucae. After one day, the foil is removed. Experiments were performed in triple, and eight to ten days after infection leaves are phenotypically scored by eye on the presence of Bremia, i.e. being susceptible or resistant.

Disease resistance tests show that the V10 resistance gene provides resistance to Bremia races from Bl:16EU to Bl:37EU (See FIG. 2). Furthermore, disease resistance test show that the V10 resistance gene further provides resistance to US Bremia races Bl:1 to Bl:9 (results not shown). Previous disease resistance test have shown that the V10 resistance gene also provides resistance to Bl:1EU to Bl:15EU (results not shown), therefore the V10 resistance gene provides full spectrum resistance to Bl:1-37EU.

A single gene line comprising the V10 resistance gene was used internally to test Bremia diagnostically. Seeds of this line are deposited at NCIMB Ltd, Aberdeen, Scotland on 12 Jul. 2017 under the number NCIMB 42786.

1. A downy mildew resistant lettuce plant, wherein said lettuce plant comprises a V10 resistance gene encoding a protein that provides downy mildew resistance and has at least 95% amino acid sequence identity with SEQ ID NO: 2 wherein said lettuce plant is resistant to at least one of Bremia lactucae races Bl:16-37EU and Bl:1-9US.

2. The lettuce plant according to claim 1, wherein said V10 resistance gene comprises a coding sequence having at least 95% sequence identity with SEQ ID NO: 1.

3. The lettuce plant according to claim 1, wherein the lettuce plant is further resistant to one or more of Bremia lactucae races selected from the group consisting of races Bl:1-15EU.

4. The lettuce plant according to claim 1, wherein the lettuce plant is selected from the group consisting of Lactuca sativa, Lactuca virosa, Lactuca saligna, Lactuca serriola, Lactuca aculeate, Lactuca georgica, Lactuca perennis, Lactuca tatarica, and Lactuca viminea, preferably Lactuca sativa.

5. The lettuce plant according to claim 1, wherein the V10 resistance gene is obtainable, derived, or originates from a lettuce plant deposited under number NCIMB 42786.

6. A seed of the lettuce plant according to claim 1, the seed comprising the V10 resistance gene.

7. A resistance gene that confers resistance to downy mildew in lettuce plants, wherein the resistance gene encodes for a protein that has at least 95% sequence identity with SEQ ID NO: 2.

8. The resistance gene according to claim 7, wherein a coding sequence of said resistance gene has at least 95% sequence identity with SEQ ID NO: 1.

9. The resistance gene according to claim 7, wherein the resistance gene provides resistance to at least Bremia lactucae races Bl:16 to Bl:37.

10. A method for identifying (i) the downy mildew resistant lettuce plant according to claim 1 or (ii) a seed of said plant, the method comprises the step of establishing, in the genome of a plant or seed the presence of the V10 resistance gene.

11. The method according to claim 10, wherein the step of establishing comprises establishing the presence of SEQ ID NO: 14.

12. A method for providing a lettuce plant that is resistant to downy mildew, wherein the method comprises the steps of:
(a) crossing a lettuce plant comprising the resistance gene according to any one of the claim 7 with a lettuce plant that is susceptible to downy mildew and does not comprise said resistance gene, and
(b) selecting plants that are resistant to downy mildew.

13. A method of introducing a resistance gene into the genome of a plant or plant cell and providing broad spectrum resistance to downy mildew caused by one or more of B. lactucae selected from the group of race Bl:1-37EU and/or BL1-9US, wherein the method comprises uses of a gene construct or plasmid of comprising the resistance gene according to claim to operably linked to expression providing sequences in said plant.

14. The lettuce plant according to claim 1, wherein said lettuce plant is resistant to Bremia lactucae races Bl:16-37EU and Bl:1-9US.

15. The lettuce plant according to claim 1, wherein the lettuce plant is Lactuca sativa.

16. The resistance gene according to claim 7, wherein the resistance gene provides resistance to Bremia lactucae races Bl:1-37EU or Bl:1-9US in lettuce.

17. The resistance gene according to claim 7, wherein the resistance gene provides resistance to Bremia lactucae races Bl:1-37EU and Bl:1-9US in lettuce.

18. The method according to claim 10, wherein the step of establishing comprises establishing the presence of SEQ ID NO: 3 in combination with SEQ ID NO: 4.

19. The method according to claim 10, wherein the step of establishing comprises establishing the presence of SEQ ID NO: 1 in combination with SEQ ID NOS: 3 and 4.

20. The method according to claim 12, further comprising selfing the plant obtained in step (a) for at least one time, prior to the step of (c) selecting plants that comprise said stay green gene or nucleic acid sequence.


SEQUENCE LISTING (6 sequences)

SEQ ID NO: 1 - DNA, 2943 bp, Lactuca virosa
  cDNA
atggttgttttatctgaacttccagaaggatcttcatattcttcatcaactcatggccatagtccatcaacttgtggtccaagttcatcaacccatgctcatagatatgatgtatttctaagttttagaggtctcgatactcgtcatagtttcactgatcaccttcataaggctctcattgatgccaatatcactaccttcttggatgatgaagagatagaaacaggggaagatctgaaaccggaattagagagtgcaattaaggcatctcgggcttctattatcatcttgtctaagaattatgcttcttcaacatggtgcctggatgaactggtgctgatccttgagcagcgtatgacatctgatcatattgtcatccccatcttttatcatgtggagcccacccatgtcaggaagcagcaaagtagctttggagatgcaatggctaagcataaacaaacaatggaggcagagacaaatgcaaataaaagaagtcaatggggtcaaaagatagagagatggaataaagcacttatcgaagttgctaatttaaaaggaaatgacatcaatggaaggtttgagacagagtttattgaagaaattgtgaaggacatctaccgtagattacatgtacccttaaaaagtgttcaacctctatttattgggatggactattccattaacttcataacttcatggttgaaagatggatcctcacatacggtagacatactcaccgtttcgggtataggcgggatcgggaagacatctttagccaaacatgtctatgggctatattggcatgaattccacaaaagcagctatattgaagatatcactaggagatgtgctggaaagtataacgggctgcttgatatacaaaaacaactgtgcggtgacatttcaaaaacaagttcaattcaagtttatgatgtttcaaaatacacatcaatgatagagaatgcagtagctcgtaaaaaggtgtttcttgttcttgatgatatcaatagtattgatcagttggatgcattacttggaagcaaaggttttcatcctggaagcaaggttataataaccacaaaggacagatgcttgacagagagttgtgcactattcaaaacaaacagtaaagccaaacatgtagaacacttccttaaaggcttatctcagactaaatcacaacaacttttgtggtcccatgcattcatgtgcaaccatcccatggaaggttatgaacaggtttcagataagcttgtggagtattgtcaaggacatccattggctcttgaagttttgggcaagcttcttcataatcgagatgtggcgtattgggaagggtgcatgaaagggctaaagaaagaaacaaatgatcgtataaataatgtgttgagaatgagcttcgactctttgccatccaaaaacgataaggacttgtttaagcatattgcttgtttttttgttggaatagatagagatcttagcgaaacaatattggaggcatgtgatataaacacgacatccggaataacgaatctcattgacagatgtcttcttagtattggatgggagaatgaattgaagatgcatcagttggttcaagaaatgggaaaattcgaagtacatcaagaatcacctgataagccatggaagcgaagtcggttatggtgtcataaggagtcattcagagtgttgaaacaaaaaaagggtaagggaaatcttctaggccttgcccttgacatgcgcatgcttgagaaagagaagttgggagcatcgtatgagctgaaaacagatgcattaagtaacatggataatttgatgctactacaactcaattatgtgtacatgaatgggtcttatgagaaatttccagaagaaataaaatggttgtgtatgaatgggtttcatttaaagtctatacctttagacttacctatgcagaatctggttgcccttgacatgtcatatagcaatattgaatcatttgtcggttgttatagcaatccacaacgacttgagaagaggcaaaagttggatggctcatgcttaaaagagaaaaggttgtttggttcactgaagattcttaatttaagtttctgcacacaacttcatagtattggtgattttgaccaactccctgcgcttgagaggttaatacttagaaattgcattcgtttggttgatgtttgtgaatcaattgggcaatgtgttagacttatcctcatcgatctgagagactgcatgaaacttgaaaaacttccaagaaatataggcatgttaaacaaggttgaaacaatgttgttagatggttgtaatcttggtgaatctcgaatcaagaatatggatatggattcactggaaatgtgcacggttaccaacattggcataaacagagtctttatgggttgtataccacgtgatttgaagtcctctgcaatgtctttaccgaggtctttagtaacattgtcactcacaaagaacaagttgtccaatgaatcctttcccttggacttcagttgtctatccatgttaaaggaattatatttagatggcaatcttttcaattccatgcctagttgtgtgagaacccttcctagacttgagatacttagtatgaataagtgtaaaaatttgaagtcagtcgagtatcctccacgtacattaaaagagttgtttcttattgctagtgatgattatcatacagacaaagttgtatttgttccagaaatgtccccactccagttatcaatggactggttagactggatatcttcttcacttgggttgaacgattatgaaattgaaggtatgatcaaaatacaaaaaatgatgagtgttgatgaaaaggtgttacatagcttgggctggactaatttagacttcctcaacgaaagacacgtggtaactaattcttcagaatctgaaatccaggttttgtctagttaa

SEQ ID NO: 2 - protein, 980 aa, Lactuca virosa
  protein sequence
MVVLSELPEGSSYSSSTHGHSPSTCGPSSSTHAHRYDVFLSFRGLDTRHSFTDHLHKALIDANITTFLDDEEIETGEDLKPELESAIKASRASIIILSKNYASSTWCLDELVLILEQRMTSDHIVIPIFYHVEPTHVRKQQSSFGDAMAKHKQTMEAETNANKRSQWGQKIERWNKALIEVANLKGNDINGRFETEFIEEIVKDIYRRLHVPLKSVQPLFIGMDYSINFITSWLKDGSSHTVDILTVSGIGGIGKTSLAKHVYGLYWHEFHKSSYIEDITRRCAGKYNGLLDIQKQLCGDISKTSSIQVYDVSKYTSMIENAVARKKVFLVLDDINSIDQLDALLGSKGFHPGSKVIITTKDRCLTESCALFKTNSKAKHVEHFLKGLSQTKSQQLLWSHAFMCNHPMEGYEQVSDKLVEYCQGHPLALEVLGKLLHNRDVAYWEGCMKGLKKETNDRINNVLRMSFDSLPSKNDKDLFKHIACFFVGIDRDLSETILEACDINTTSGITNLIDRCLLSIGWENELKMHQLVQEMGKFEVHQESPDKPWKRSRLWCHKESFRVLKQKKGKGNLLGLALDMRMLEKEKLGASYELKTDALSNMDNLMLLQLNYVYMNGSYEKFPEEIKWLCMNGFHLKSIPLDLPMQNLVALDMSYSNIESFVGCYSNPQRLEKRQKLDGSCLKEKRLFGSLKILNLSFCTQLHSIGDFDQLPALERLILRNCIRLVDVCESIGQCVRLILIDLRDCMKLEKLPRNIGMLNKVETMLLDGCNLGESRIKNMDMDSLEMCTVTNIGINRVFMGCIPRDLKSSAMSLPRSLVTLSLTKNKLSNESFPLDFSCLSMLKELYLDGNLFNSMPSCVRTLPRLEILSMNKCKNLKSVEYPPRTLKELFLIASDDYHTDKVVFVPEMSPLQLSMDWLDWISSSLGLNDYEIEGMIKIQKMMSVDEKVLHSLGWTNLDFLNERHVVTNSSESEIQVLSS

SEQ ID NO: 3 - DNA, 121 bp, Artificial Sequence
  Marker 1
actagcatataaaaagcataacagatagtggcataccagacaattatcacaaagacaatcatctacatataaacaagtactagtgggccggcattggtgccttagacccacatctaccgga

SEQ ID NO: 4 - DNA, 121 bp, Artificial Sequence
  Marker 2
ccataaattccccgtcgccgggaccttccccattgattcaaccccaacaccctatccctcatccaccaatttcaccaccaccaccaccgccacccatctctatacgatcaccgcctcctcc

SEQ ID NO: 5 - DNA, 300 bp, Artificial Sequence
  VIGS construct B4
acaaaagcagctatattgaagatatcactaggagatgtgatggaaagtatattgggctgcttgatatacaaaaacaactgtgtggtgatatttcaaaaacaagttcaattcaaatttatgatgtttcaaaatacacatcaatgatagagaatgcagtagctcgtaaaagggtgtttattgttcttgatgatatcaatagtattgatcagttggatgcattacttggaagcaaaggttttcatcctggaagcaaggttataataaccacaaaggacagatgcttgacagagagttgtgcac

SEQ ID NO: 6 - DNA, 250 bp, Artificial Sequence
  VIGS construct F12
tggcggcactacatccaccatatgtgtttgtatgcctcttcttcgtatttccaattcccacaatcatcttcctgatccccttcctgttcttggtcgcgccattgctggttgctccaatcataacagcgtcggagagaccttagcccgagatacactggaacaggtgatgatggaagccctttcaacgtcaggctcaaagaaatccgctgttcaagctgtttgtttaggcgtatcaggtgtcaatcatcca